(19) United States
(12) Patent Application Publication
(10) Pub. No.: US 2018/0147247 A1
(43) Pub. Date: May 31, 2018

(54) METHOD FOR PREPARATION OF A CANNABINOID EXTRACT FROM HEMP

(71) Applicant: "POBELCH - GLE" OOD, Sofia (BG)

(72) Inventor: Ervin Milenov IVANOV, Dulovo (BG)

(73) Assignee: "POBELCH - GLE" OOD, Sofia (BG)

(21) Appl. No.: 15/575,911

(22) PCT Filed: May 19, 2016
(86) PCT No.: PCT/BG2016/000016

§ 371 Date: Nov. 21, 2017

(30) Foreign Application Priority Data
May 22, 2015 (BG) 112018

(51) Int. Cl. A61K 36/185; A61K 31/352; A61K 31/192; A61P 29/00; A61P 31/00; A61P 3/00; A61P 25/14; A61P 25/16; A61P 25/22; A61P 25/06; A61P 19/02; A61P 27/06; A61P 25/24; B01D 11/04

(52) U.S. Cl. CPC A61K 36/185; B01D 2011/002; A61K 31/192; A61P 29/00; A61P 31/00; A61P 3/00; A61P 25/14; A61P 25/16; A61P 25/22; A61P 25/06; A61P 19/02; A61P 27/06; A61P 25/24; B01D 11/048; B01D 11/0407; A61K 2236/53; A61K 2236/55; A61K 2236/15; A61K 2236/17; A61K 2236/39; A61K 2236/37; A61K 2236/333; A61K 2236/331; A61K 2236/33; A61K 2236/31; A61K 31/352

(57) ABSTRACT

The invention relates to a method for extraction and separation of cannabinoids from industrial hemp, designed for medicinal purposes, and also the preparation of an extract, not containing tetrahydrocannabinol, and the preparation of maximum refined individual cannabinoids.

The advantage of the method according to the invention consists in the preparation of an extract from hemp, which contains at a high percentage medically useful cannabinoids and doesn't contain undesirable admixtures and tetrahydrocannabinol, so that it can be used without any restrictions as a pharmaceutical. Moreover, the method allows the possibility of separation, if required, into individual useful cannabinoids as pure compounds, in ecological terms, without environmental pollution, as it is according to the most synthetic methods. The possibility of producing pure compounds represents a great contribution to the research of substances, related to a concrete medical application and the preparation of various combinations thereof, with the objective of expansion the field of application. The method is also cost-effective.

The method consists in that the extract, obtained in accordance with various methods, undergoes a centrifugal countercurrent liquid-liquid chromatography, as the operation includes a centrifugation of solvents and the extract, obtained during the previous operations; the solvents form two phases, the phase, which the extract is dissolved in, is mobile, and the other one is stationary, whereby the mobile phase passes through the stationary phase, wherein several amounts of the components of the extract content are captured; this passing of the mobile phase through the stationary phase is repeated many times, until separation of the desired substances, which are analyzed in a familiar way, whereby as stationary phase solvents are used, which are selected from the group of straight-chain and branched-chain hydrocarbons, produced from crude oil, straight-chain and/or branched-chain alcohols, straight-chain and/or branched-chain ketones, straight-chain and/or branched chain carboxylic acids, straight-chain and/or branched-chain nitriles, gases in supercritical and subcritical condition, like carbon dioxide, nitrogen, nitrogen oxides, water with modified acidity with or without salts of organic and non-organic substances dissolved therein, as for example NaSO₃, carbonate compounds or mixtures of the above-mentioned solvents, and as mobile phase solvents are used, which are selected from the group of straight-chain and branched-chain hydrocarbons, produced from crude oil, straight-chain and/or branched-chain alcohols, straight-chain and/or branched-chain ketones, straight-chain and/or branched chain carboxylic acids, straight-chain and/or branched-chain nitriles, gases in supercritical and subcritical condition, like carbon dioxide, nitrogen, nitrogen oxides, water with modified acidity by organic and/or inorganic acids and bases, as well as Lewis acids and bases in the interval from 0 to 14 pH, with or without salts of organic and non-organic substances dissolved therein, as for example NaSO₃, carbonate compounds, or mixtures of the above-mentioned solvents, while the choice of the solvents between the two phases is conditional on that, they shall be different and immiscible with each other; the centrifugation revolutions and the flow speed of the mobile phase are designed (calculated or determined experimentally) depending on the total phase volume; thus at the finish of the process the tetrahydrocannabinols and/or the tetrahydrocannabinol acids are separated from the remaining cannabinoids and/or cannabinoid acids in the solution, the other cannabinoids in the process can also be separated as a pure substance, separate in a solution and the solvents are evaporated respectively, in order to obtain a pure substance.

### FIELD OF TIFF INVENTION

The invention relates to a method for preparation of a cannabinoid extract from hemp, designed for medicinal purposes, in particular the preparation of an extract, not containing tetrahydrocannabinol, and the preparation of maximum refined individual cannabinoids by means of extraction and chromatography.

### BACKGROUND OF THE INVENTION

Since thousands of years cannabis and hemp have played a central role in many societies. A lot of medicinal activities, inclusive relieving the symptoms of many diseases and conditions, are ascertained with the active ingredients of the cannabis strains, including Cannabis indica and Cannabis sativa. The plant contains about 70 cannabinoids and each of them can have therapeutic potential. The cannabinoids are compounds with 21 carbon atoms and carboxylic acids, analogues and metabolism products with 21 carbon atoms (the carboxylic acids are especially dominating in the living cells and fresh plants). The relative share of cannabinoids in the cannabis plants is greatly influenced by genetic factors and the environment. The main components usually include tetrahydrocannabinols (abbreviated as THC), cannabidiol (CBD) and cannabinol (CBN), together with smaller quantities of ingredients, like cannabichromene (CBC). The medicinal properties of the cannabis plants usually are due to the THC, the C D and to other cannabinoids, as well as to cannabinoid acids and terpenes.

The medicinal uses of cannabis and extracts thereof include analgesia of acute and chronic pain, antiemetic therapy, especially in chemotherapy and radiation, appetite stimulation and prevention of weight loss in HIV-infected and cancer patients, anti-spastic action, in particular with multiple sclerosis, muscular dystrophy and in patients with spine injuries, movement disorders, like Parkinson's disease, Huntington's disease, Tourette syndrome, epilepsy, glaucoma, depression, anxiety and stress relief, migraine, arthritis and rheumatism; supporting action in various other chronic disorders and conditions. While the researches have not yet found out, exactly which substances are responsible for these useful effects, the clinical practice indicated, that the application of plant material or total extracts has advantages over isolated or synthetic compounds, like pure THC (the Marinol preparation).

From ancient times the assimilation of useful substances takes place in different ways: through smoking or through inhalation of plant vapors. Nowadays the use of plant extracts is in the first place.

In recent years the mechanical extraction, the solvent extraction and extractions, supported by different physical methods and means, are used in the field of preparation of plant extracts. The patent EP 1 326 598 B1 “Method for preparation of extracts, containing tetrahydrocannabinol and cannabidiol from plant material cannabis, as well as an extract from cannabis” discloses a method for preparation of an extract, containing tetrahydrocannabinol, cannabidiol and possibly their carboxylic acids from cannabis plant material, whereby dry plant matter is extracted with CO₂ under conditions of supercritical pressure and temperature, at a temperature within the limits from 31 to 80° C. and a pressure within the limits from 75 to 500 bar, or it is extracted within supercritical temperature limits from 20 to 30° C. and supercritical pressure limits from 100 to 350 bar; and the obtained first extract is separated under supercritical conditions. A sucking gas agent, selected from propane, butane, ethanol and water, is added to the CO₂. An adsorbent layer, located in the direction of the CO₂ flow, is put on the extracted material. The adsorbent is selected among silica gel, diatomite (diatomaceous earth) bentonite, chlorinated lime, active carbon, in particular magnesium oxide and aluminum oxide, or a composite thereof. The first cannabis extract, containing tetrahydrocannabinol and cannabidiol, and possibly their carboxylic acids, is characterized in that it comprises at least reduced quantities of monoterpenes and sesquiterpenes, alkaloids, flavonoids and chlorophyl. Tetrahydrocannabinol consists of delta-9-tetrahydrocannabinol and delta-8-tetrahydrocannabinol.

A disadvantage of this method is the high content of tetrahydrocannabinols, which causes undesirable side effects in use for medical purposes, including an opiate effect, and leads to addiction.

EP 1 385 595 B1 “Method for preparation of an extract from cannabis plant material, enriched with cannabinoids” describes a method, which includes the contact of the cannabis plant material with heated gas at a temperature from 105 to 450° C. and within a given time period, enough to separate one or more cannabinoids in the form of vapor, but without to provoke pyrolysis of the plant material and vapor condensation, for preparation of an extract, enriched with cannabinoids. The temperature is rising in two steps. For example, during processing of plant matter with a high content of cannabidiol, the first temperature range is from 125 to 150° C., and the second from 175 to 200° C. During processing of plant matter with a high content of THC, the first temperature range is from 60 to 90° C., and the second from 130 to 175° C. Basically, the extract doesn't contain dietary fiber, fats, waxes, carbohydrates, proteins and sugars, as well as terpenes. The extract also is enriched with THC.

For the purpose of obtaining an extract from cannabinoids with a low THC content, RU 2010116181 A describes a plant extract from hemp with a low content of tetrahydrocannabinol for treatment of diseases, which is obtained by extraction with a solvent and/or mixture of solvents, selected among the group: water, sodium chloride solution, lower alcohols, acetone, esters or ethers.

A combination of extraction by means of column chromatography is applied for the purpose to separate the different cannabinoids, the idea for which is given in U.S. Pat. No. 6,403,126 B1 as a method for preparation of an extract from cannabis, including collection of plant material, comprising seed and straw, separation of straw from seed, extraction of straw by means of a solvent, transportation of the extract, if required, throughout a chromatographic column, which is designed to separate delta-9-THC from the extract and collection of the fractions, which don't contain delta-9-THC from the column, whereby a total extract from hemp without delta-9-THC is obtained. Industrial hemp, which is a cannabis plant with a low content of delta-9-tetrahydrocannabinol (delta-9-THC), is used as a plant raw material. Straw means a chopped plant matter, remaining after the separation of seed, which can be green or dry. The solvent can be an organic solvent, selected among the group, comprising hydrocarbons, separated from crude oil, for example toluol, 3-methylpentane; lower alcohol, for example ethanol; lower chlorinated hydrocarbon, for example chloroform and dichloromethane; supercritical fluid like CO₂, with or without an organic modifier. The extraction can also be performed employing techniques, like extraction with increasing solvent quantity, or by means of subcritical water. This is disclosed in the description. According to another aspect of this invention, a method for extraction of cannabinoid, canflavine and volatile oil from cannabis is created, whereby the applied chromatographic column is arranged to fractionate cannabinoid, canflavine or volatile oil from the extract. The cannabinoid can be selected among the group, consisting of cannabidiol, cannabinol, cannabigerol, cannabichromene, cannabidiverol, tetrahydrocannabidiol, tetrahydrocannabigerol, tetrahydrocannabichromene, tetrahydrocannabidiverol, delta-8-tetrahydrocannabinol; the carboxylic acids of the above compounds and also natural compounds and their derivates accordingly. The claims make reference to this aspect of the invention.

As a matter of fact, this patent document doesn't disclose the features of the column, like loading, temperature conditions, flow speed, column dimensions and other parameters, in order to enable a person of ordinary skill in the art to re-create the method. The problem to be solved is the preparation of an extract from hemp, which extract to contain medically useful cannabinoids at a high percentage, but to contain none, or to contain admixtures in a minimum quantity, in that case isomers of tetrahydrocannabinol, which has opiate action and in most cases of medical use of cannabinoids this action is undesirable. Moreover, the method shall give the possibility of separation, if desired, into individual useful cannabinoids.

### DISCLOSURE OF THE INVENTION

The problem is solved by a method, which represents a combination of extraction under various methods and a subsequent centrifugal countercurrent liquid-liquid chromatography. All hemp strains can be used for the purposes of this method; one hemp strain is preferable the industrial hemp, which has genetically low content of tetrahydrocannabinol isomers. In accordance with one aspect of the invention, the extraction takes place as dry and/or fresh plant matter, and/or mechanical extract, and/or chemical extract from industrial hemp (Cannabis sativa L.) is extracted non-selectively or selectively, using solvents under supercritical or subcritical conditions from the group of carbon dioxide, pure water, water with modified acidity with or without salts of organic and non-organic substances dissolved therein like, for example, sodium stearate, NaSO₃, carbonate compounds and others, nitric oxides, nitrogen, hydrocarbons, separated from crude oil, for example pentane, hexane, toluol, 3-methylpentane; higher and lower alcohols, ketones, nitrile compounds, halogenated hydrocarbons, separated from crude oil, and/or mixture of these solvents, with or without cosolvents from the group of hydrocarbons, separated from crude oil, for example pentane, hexane, toluol, 3-methylpentane, higher and lower alcohols, ketones, nitrile compounds, halogenated, hydrocarbons, separated from crude oil and/or the relative mixture thereof, at temperatures from −20 to 380° C. and at a pressure from 10 to 1000 bar, while the process can take place through soaking from 1 min to [symbol] 24 hours, or through continuous processing with fresh and/or recirculating solvent. In some cases the pressure of the solution is decreased in a separate vessel. If required, a winterization is carried out, in order to separate the waxes. At the next step, the pressure of the extract solution, obtained at the previous stage, is reduced until separation of the cannabinoids and/or the cannabinoid acids, and the related substances of the solvent in the form of resin, including in some cases volatile substances. If required, the volatile substances are separated at the next stage by heating from 5 to 300° C. or freezing from −100 to 5° C. by means of evacuating or creating a subatmospheric pressure, while the volatile substances in the flow are either condensed, or transported throughout pure water, water with modified acidity with or without salts of organic and non-organic substances dissolved therein like, for example, sodium stearate, NaSO₃, carbonate compounds and others, and/or an organic solvent from the group of hydrocarbons, separated from crude oil, for example pentane, hexane, toluol, 3-methylpentane, higher and lower alcohols, ketones, nitrile compounds, halogenated hydrocarbons and/or mixture of these solvents.

According to another aspect of the invention, the extraction is performed with solvents from the group, consisting of hydrocarbons separated from crude oil, for example pentane, hexane, toluol, 3-methylpentane; lower and higher alcohols, for example ethanol, isopropyl; ketones, for example acetone; nitrile compounds, for example acetonitrile; halogenated hydrocarbons, for example chloroform and dichloromethane, under application of soaking at intervals from 1 min to 24 hours; flushing, whereby the solvent and the plant matter are in contact for a few seconds; reflux or ultrasound assisted extraction for a time period from 1 min up to 24 hours.

In accordance with another aspect of the invention, a mechanical extract is produced through collection from the plant surface of resin and trichomes, which contain useful cannabinoids in quantity. A mechanical extract can also be obtained by means of freezing or desiccation of trichomes and also chopping, screening and blowing.

Depending on the method of extraction and due to the fact, that an extract with different content of undesirable and/or targeted components can be obtained, alternative intermediate operations take place before the next operation chromatography, for example: decarboxylation with neutral cannabinoid as a targeted component, winterization in the presence of undesirable waxes; liquid-liquid extraction in the presence of water-soluble undesirable substances in the extract; flushing through an adsorbent and/or molecular sieve, and/or glass material, and/or sand in the presence of polar and/or non-polar undesirable, and/or targeted substances; filtration or a combination thereof.

The extract, obtained in accordance with some of the above-mentioned aspects of the invention, undergoes a centrifugal countercurrent liquid-liquid chromatography and particularly centrifugal partition chromatograpy (CPC).

This operation includes a centrifugation of solvents and the extract, obtained during the previous operations. The solvents form two phases. The phase, which the extract is dissolved in, is mobile, and the other one is stationary, whereby the mobile phase passes through the stationary phase, wherein several amounts of the components of the extract content are captured and/or delayed. This passing of the mobile phase through the stationary phase is repeated many times, until separation of the desired substances.

As stationary phase solvents are used, selected from the group of straight-chain and branched-chain hydrocarbons, produced from crude oil, straight-chain and/or branched-chain alcohols, straight-chain and/or branched-chain ketones, straight-chain and/or branched chain carboxylic acids, straight-chain and/or branched-chain nitriles, solvents in supercritical or subcritical condition, like methane, propane, butane, carbon dioxide, nitrogen, nitrogen oxides, pure water, water with modified acidity with or without salts of organic and non-organic substances dissolved therein, as for example sodium stearate, NaSO₃, carbonate compounds or mixtures of the above-mentioned solvents. As a mobile phase solvents are used, selected from the group of straight-chain and branched-chain hydrocarbons, produced from crude oil, straight-chain and/or branched-chain alcohols, straight-chain and/or branched-chain ketones, straight-chain and/or branched chain carboxylic acids, straight-chain and/or branched-chain nitriles, pure water, as well as water with modified acidity, with or without salts of organic and non-organic substances dissolved therein, as for example sodium stearate, NaSO₃, carbonate compounds, solvents in supercritical or subcritical condition, like methane, propane, butane, carbon dioxide, nitrogen, nitrogen oxides, pure water, as well as water with modified acidity, with or without salts of organic and non-organic substances dissolved therein, as for example sodium stearate, NaSO₃, carbonate compounds or mixtures of the above-mentioned solvents. The choice of the solvents between the two phases is conditional on that, they shall be different and immiscible with each other.

The centrifugation revolutions and the flow speed of the mobile phase are designed (calculated or determined experimentally) depending on the total phase volume. These parameters belong to the major parameters when designing CPC for implementation of the method, or when selecting CPC, available on the market. At the finish of the process the tetrahydrocannabinols and/or the tetrahydrocannabinol acids are separated from the remaining cannabinoids and/or cannabinoid acids in a solution. The other cannabinoids in the process can also be separated as a pure substance, separate in a solution. The solvents are evaporated respectively, in order to obtain a pure substance. The process can be repeated for refining The advantage of the method according to the invention consists in the preparation of an extract from hemp, which contains at a high percentage medically useful cannabinoids and doesn't contain undesirable admixtures and tetrahydrocannabinol, so that it can be used without any restrictions as a pharmaceutical. Moreover, the method allows the possibility of separation, if required, into individual useful as pure compounds, in ecological terms, without environmental pollution, as it is according to the most synthetic methods. The possibility of producing pure compounds represents a great contribution to the research of substances, related to a concrete medical application and the preparation of various combinations thereof, with the objective of expansion the field of application. The method is also cost-effective.

### BRIEF EXPLANATION OF THE FIGURES

FIG. 1: HPLC of plant matter.

FIG. 2: Fraction sequence.

FIG. 3: HPLC of integrated pure fractions.

Table 1: Molar solubility of THC at different temperatures and pressure in supercritical carbon dioxide.

Table 2: Molar solubility of CBD at different temperatures and pressure in supercritical carbon dioxide.

Table 3: Molar solubility of CBG at different temperatures and pressure in supercritical carbon dioxide

Table 4: Molar solubility of CBN at different temperatures and pressure in supercritical carbon dioxide

### EXEMPLARY EMBODIMENT 1: ISOLATION OF THC FROM A TOTAL HEMP EXTRACT

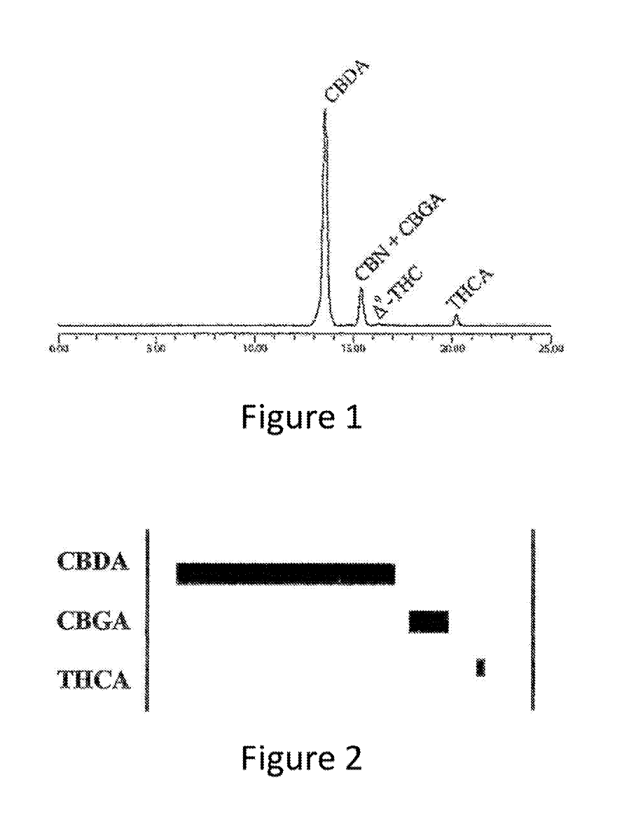

In accordance with this embodiment the industrial hemp is harvested and the seed is separated from the straw. The straw is collected and it can be desiccated or fresh extracted. According to the example described here, the straw is desiccated, separated from the seeds, chopped and pulverised. This embodiment requires 1000 g leaf and fruit tips, as well as flower heads of industrial hemp containing tetrahydrocannabinol (THC) 0.06%, tetrahydrocannabinol acid (THCA) 0.01%, cannabidiol acid (CBDA) 1,16%, cannabigerol acid (CBGA) 0.1%, cannabinol (CBN) 0.1% (FIG. 1), and is aimed at the isolation of the psychoactive THC and the respective acid form from the remaining components of the hemp extract.

Also, the given plant matter contains a low amount of THC, which can be extracted with a determined quantity of SCCD, so that only CBD, CBG and CBN, which will be extracted in a second step for preparation of a hemp extract without THC, remain in the plant matter.

TABLE 1
Molar solubility of THC at different temperatures
and pressure in supercritical carbon dioxide

| t = 40° C. | | t = 50° C. | | t = 60° C. | |
|---|---|---|---|---|---|
| P (bar) | 10₄y | P (bar) | 10₄y | P (bar) | 10₄y |
| 130 | 0.20 ± 0.02 | 130 | 0.26 ± 0.03 | 130 | 0.31 ± 0.03 |
| 190 | 0.66 ± 0.07 | 150 | 0.44 ± 0.04 | 150 | 0.70 ± 0.07 |
| 200 | 0.66 ± 0.07 | 170 | 0.71 ± 0.07 | 170 | 1.56 ± 0.16 |
| 230 | 0.71 ± 0.07 | 190 | 0.76 ± 0.08 | 200 | 1.66 ± 0.17 |
| 250 | 0.81 ± 0.08 | 200 | 1.32 ± 0.13 | 220 | 2.12 ± 0.21 |
| | | 220 | 1.41 ± 0.14 | 230 | 2.85 ± 0.29 |
| | | 230 | 2.00 ± 0.20 | | |

TABLE 2
Molar solubility of CBD at different temperatures and
pressure in supercritical carbon dioxide

| t = 40° C. | | t = 50° C. | | t = 60° C. | |
|---|---|---|---|---|---|
| P (bar) | 10₄y | P (bar) | 10₄y | P (bar) | 10₄y |
| 110 | 1.00 ± 0.10 | 120 | 0.95 ± 0.09 | 115 | 0.90 ± 0.09 |
| 120 | 1.20 ± 0.12 | 125 | 1.60 ± 0.16 | 120 | 1.20 ± 0.12 |
| 125 | 1.30 ± 0.13 | 130 | 1.89 ± 0.19 | 125 | 1.61 ± 0.16 |
| 130 | 1.30 ± 0.13 | 135 | 1.88 ± 0.19 | 130 | 1.85 ± 0.18 |
| 140 | 1.42 ± 0.14 | 140 | 2.22 ± 0.22 | 145 | 1.75 ± 0.17 |
| 145 | 1.61 ± 0.16 | 155 | 2.50 ± 0.25 | 160 | 2.00 ± 0.20 |
| 155 | 1.70 ± 0.17 | 165 | 2.69 ± 0.27 | 165 | 1.82 ± 0.18 |
| 160 | 1.63 ± 0.16 | 195 | 2.71 ± 0.27 | 170 | 1.75 ± 0.17 |
| 170 | 1.89 ± 0.19 | | | | |
| 175 | 1.89 ± 0.19 | | | | |

TABLE 3
Molar solubility of CBG at different temperatures and
pressure in supercritical carbon dioxide

| t = 40° C. | | t = 50° C. | | t = 60° C. | |
|---|---|---|---|---|---|
| P (bar) | 10₄y | P (bar) | 10₄y | P (bar) | 10₄y |
| 130 | 1.15 ± 0.12 | 130 | 1.20 ± 0.12 | 130 | 1.31 ± 0.13 |
| 155 | 1.20 ± 0.12 | 150 | 1.21 ± 0.12 | 150 | 1.39 ± 0.14 |
| 175 | 1.19 ± 0.12 | 160 | 1.21 ± 0.12 | 170 | 1.60 ± 0.16 |
| 185 | 1.23 ± 0.12 | 165 | 1.33 ± 0.13 | 180 | 1.79 ± 0.18 |
| 195 | 1.30 ± 0.13 | 170 | 1.36 ± 0.14 | 185 | 1.80 ± 0.18 |
| 200 | 1.30 ± 0.13 | 180 | 1.34 ± 0.13 | 190 | 1.92 ± 0.19 |
| 205 | 1.50 ± 0.15 | 190 | 1.45 ± 0.14 | 200 | 2.01 ± 0.20 |
| 210 | 1.60 ± 0.16 | 200 | 1.53 ± 0.15 | 210 | 2.08 ± 0.21 |
| | | 210 | 1.69 ± 0.17 | | |

TABLE 4
Molar solubility of CBN at different temperatures
and pressure in supercritical carbon dioxide

| t = 40° C. | | t = 50° C. | | t = 60° C. | |
|---|---|---|---|---|---|
| P (bar) | 10₄y | P (bar) | 10₄y | P (bar) | 10₄y |
| 130 | 1.23 ± 0.12 | 130 | 2.43 ± 0.24 | 130 | 2.41 ± 0.24 |
| 140 | 1.28 ± 0.13 | 135 | 3.00 ± 0.30 | 135 | 2.30 ± 0.13 |
| 145 | 1.30 ± 0.13 | 140 | 3.69 ± 0.37 | 150 | 3.01 ± 0.30 |
| 150 | 1.46 ± 0.15 | 145 | 3.69 ± 0.37 | 155 | 3.31 ± 0.33 |
| 155 | 1.61 ± 0.16 | 150 | 3.80 ± 0.38 | 160 | 3.29 ± 0.33 |
| 160 | 1.80 ± 0.18 | 155 | 3.90 ± 0.39 | 170 | 3.50 ± 0.35 |
| 170 | 1.92 ± 0.19 | 160 | 3.90 ± 0.39 | 180 | 3.20 ± 0.32 |
| 180 | 2.01 ± 0.20 | 170 | 4.50 ± 0.45 | 200 | 3.20 ± 0.32 |
| 190 | 2.16 ± 0.22 | 180 | 4.25 ± 0.43 | | |
| 200 | 2.34 ± 0.23 | | | | |

The chopped straw is heated to 105° C. for 50 min and is extracted. In the first step 1 kg SCCD at 170 bar and 60° C. is used, and in the second step 10 kg SCCD at 130 bar and 40° C. are used. During the next step the solvent pressure is reduced to 50 bar in a separate vessel. Subsequently, the carbon dioxide is separated from the obtained extract by means of depressurization. The extract is heated to 40° C. under vacuum 10 torr and the volatile substances are isolated. The obtained extract is dissolved in the organic solvent ethanol in a ratio of 1 kg:10 l and it is freezed at −20° C. The coagulated waxes are separated by filtration and the liquid extract is concentrated by evaporation. 510 mg extract from the first step is dissolved to 5 ml in hexane. The dissolved extract undergoes a centrifugal partition chromatography (CPC), which is a type of countercurrent liquid-liquid chromatography. In this particular case a filler for a solvent system with capacity 100 ml is used. The selected system is hexane/acetone/acetonitrile in volume ratios 5:2:3. The process takes place in an ascending mode, while acetone/acetonitrile is used as stationary phase, and hexane as mobile phase. The flow speed is 5 ml/min and the rotation speed is 600 rpm. The dissolved extract is injected into the system. Fractions of 5 ml each are collected and the fractions, containing THC, are separated, while the remaining fractions are integrated. The fractions, containing other cannabinoids and THC, undergo a CPC in the same way, until pure THC is isolated. All fractions except THC are integrated. They are concentrated and the yield represents some percentage; subsequently the obtained substance is desiccated and/or dissolved in a new solvent and thus a total extract from hemp without THC is obtained as a result.

### Exemplary Embodiment 2: Preparation of Pure Substances

According to this embodiment the industrial hemp is harvested and the seed is separated from the straw. The straw is collected and it can be desiccated or fresh extracted. In accordance with the example described here, the straw is desiccated, separated from the seeds, chopped and pulverised. The chopped straw is extracted. This embodiment requires 100 g leaf and fruit tips, as well as flower heads of industrial hemp according to Example 1. In that case the isolation of cannabinoid acids as pure substances is targeted.

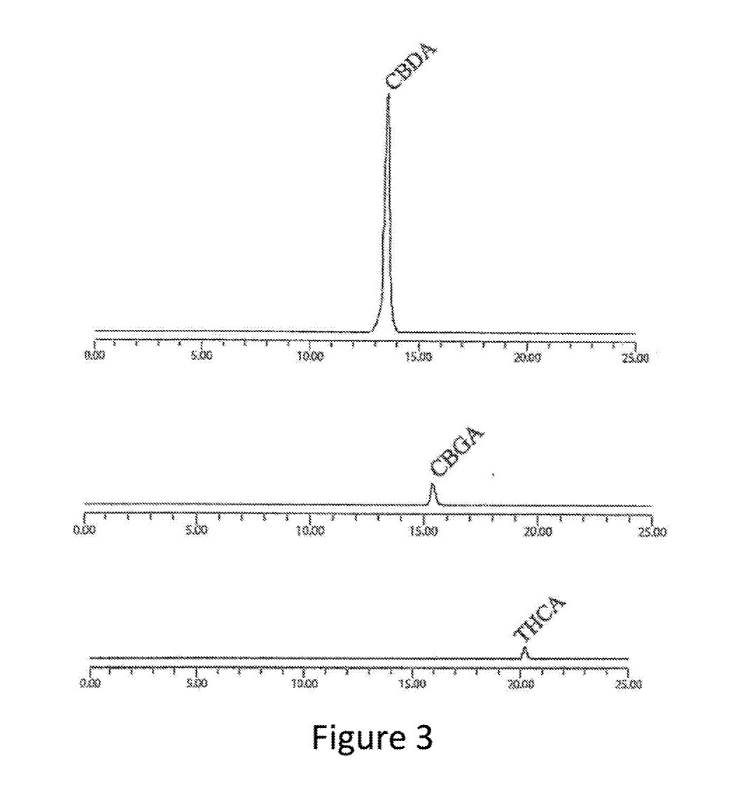

Pentane is used as a solvent at room temperature. 100 g chopped straw is soaked in the solvent in ratio of 1 kg:15 l and is let to stay for 24 hours, while it is mixed every hour. The solvent is percolated from the straw and it is evaporated under reduced pressure until the preparation of 5 ml solution. It is put on a filter bed of glass filter, filed with sand. The extract is flushed through a water solution of 0.2 M NaOH until the full washing out of the saturated color and the streaming of a clear spurt of the solution, whereby the cannabinoid acids in the water solution are isolated from the neutral cannabinoids. A hydrochloric acid is added to the solution up to pH=1.9 in order to precipitate the acid cannabinoids, which are filtrated. 2.5 g cannabinoid acids, obtained from the last process, are dissolved in 5 ml methanol. This dissolved extract undergoes a centrifugal partition chromatography (CPC), which is a type of countercurrent liquid-liquid chromatography. In this particular case a filler for a solvent system with capacity 100 ml is used. The partition system is hexane/methanol/water in volume ratios 5:3:2. The water is acidified by 0.25 mM formic acid. The process takes place in a descending mode, while methanol/water is used as mobile phase, and hexane as stationary phase. The flow speed is 4 ml/min and the rotation speed is 500 rpm. The dissolved extract is injected into the system. Fractions of 10 ml each with pure substances are collected in a solution (FIG. 2). Fractions with more than one component undergo the process once again. The fractions are analysed by means of thin-layer chromatography, uniform fractions are integrated and concentrated, and desiccated and/or dissolved in a new solvent, and thus pure cannabinoid acids are obtained as a result (FIG. 3).

1. Method for preparation of a cannabinoid extract from hemp, in particular the preparation of an extract, not containing tetrathydrocannabinol, and the preparation of maximum refined individual cannabinoids by means of extraction and chromatography, characterized in that the extract, obtained according to different methods, undergoes a centrifugal countercurrent liquid-liquid chromatography, and the operation comprises a centrifugation of solvents and the extract obtained during the previous operations, the solvents form two phases, the phase, in which the extract is solved, is mobile, and the other one is stationary, whereby the mobile phase pass throughout the stationary phase, wherein some quantifies of the components, contained in the extract, are captured and/or delayed, this passing of the mobile phase through the stationary phase is repeated many-times until separation of the desired substances, whereby as a stationary phase solvents are used, which are selected from the group of straight-chain and branched-chain hydrocarbons, produced from crude oil, straight-chain and/or branched-chain alcohols, straight-chain and/or branched-chain ketones, straight-chain and/or branched chain carboxylic acids, straight-chain and/or branched-chain nitriles, solvents in supercritical or subcritical condition, like methane, propane, butane, carbon dioxide, nitrogen, nitrogen oxides, pure water, water with modified acidity with or without salts of organic and non-organic substances dissolved therein, as for example sodium stearate, NaSO₃, carbonate compounds or mixtures of the above-mentioned solvents, and as a mobile phase solvents ate used, selected horn the group of straight-Chain and branched-chain hydrocarbons, produced from crude oil, straight-chain and/or branched-chain alcohols, straight-chain and/or branched-chain ketones, straight-chain and/or branched chain carboxylic acids, straight-chain and/or branched-chain nitriles, pure water, as well as water with modified acidity with or without salts of organic and non-organic substances dissolved therein, as for example sodium stearate, NaSO₃, carbonate compounds, solvents in supercritical or subcritical condition, like methane, propane, butane, carbon dioxide, nitrogen, nitrogen oxides, pure water, as well as water with modified acidity, with or without salts of organic and non-organic substances dissolved therein, as for example sodium stearate, NaSO₃, carbonate compounds or mixtures of the above-mentioned solvents, while the choice of the solvents between the two phases is conditional on that, they shall be different and immiscible with each other, the centrifugation revolutions and the flow speed of the mobile phase are designed (calculated or determined experimentally) depending on the total phase volume, at the finish of the process the tetrahydrocannabinols and/or the tetrahydrocannabinol acids are separated from the remaining cannabinoids and/or cannabinoid acids in the solution, and the other cannabinoids in the process can also be separated as a pure substance separate in a solution. The solvents are evaporated respectively, in order to obtain a pure substance. The process can be repeated for refining.

2. Method according, to claim 1, characterized in that extraction takes place while dry and/or fresh plant matter, and/or mechanical extract, and/or chemical extract from industrial hemp Cannabis stiva L. is extracted non-selectively or selectively using solvents under supercritical or subcritical conditions from the group of carbon dioxide, pure water, water with modified acidity, with or without salts of organic and non-organic substances dissolved therein like, for example, sodium stearate, NaSO₃, carbonate compounds and others, nitric oxides, nitrogen, hydrocarbons, separated from crude oil, for example pentane, hexane, toluol, 3-methylpentane, higher and lower alcohols, ketones, nitrile compounds, halogenated hydrocarbons, separated from crude oil, and/or mixture of these solvents, with or without cosolvents from the group of hydrocarbons, separated from crude oil, for example pentane, hexane, toluol, 3-methylpentane, higher and lower alcohols, ketones, nitrile compounds, halogenated, hydrocarbons, separated from crude oil and/or the relative mixture thereof, at temperatures from −20 to 380° C. and at a pressure from 10 to 1000 bar, while the process can take place through soaking from 1 min to [symbol] 24 hours, or through continuous processing with fresh and/or recirculating solvent, in some cases the pressure of the solution is decreased in a separate vessel and if required, a winterization is carried out, in order to separate the waxes; at the next step, the pressure of the extract solution, obtained at the previous stage, is reduced until separation of the cannabinoids and/or the cannabinoid acids, and the related substances of the solvent in the form of resin, including in some cases volatile substances, in that case the volatile substances are separated at the next stage by heating from 5 to 300° C. or freezing from −100 to 5° C. by means of evacuating or creating a subatmospheric pressure, while the volatile substances in the flow are either condensed, or transported throughout water with modified acidity, with or without salts of organic and non-organic substances dissolved therein like, for example, sodium stearate, NaSO₃, carbonate compounds and others, and/or an organic solvent horn the group of hydrocarbons, separated from crude oil, for example pentane, hexane, toluol, 3-methylpentane, higher and lower alcohols, ketones, nitrile compounds, halogenated hydrocarbons and/or mixture of these solvents.

3. Method in accordance with claim 1, characterized in that the extraction takes place with solvents from the group, consisting of hydrocarbons, separated from crude oil, for example pentane, hexane, toluol, 3-methylpentane; lower and higher alcohols, for example ethanol, isopropyl; ketones, for example acetone; nitrile compounds, for example acetonitrile; halogenated hydrocarbons, for example chloroform and dichloromethane, under application of soaking at intervals from 1 min to 24 hours; flushing, whereby the solvent and the plant matter are in contact for a few seconds reflux or ultrasound assisted extraction for a time period from 1 min. up to 24 hours.

4. Method according to claim 1, characterized in that the mechanical extract is produced through collection from the plant surface of resin and trichomes, which contain useful cannabinoids in quantity.

5. Method in accordance with claim 1, characterized in that the mechanical extract is obtained by means of freezing or desiccation of trichomes and chopping, and/or screening, and/or blowing.

6. Method according to claim 1, characterized in that depending of the method of extraction and due to the fact, that an extract with different content of undesirable and/or targeted components can be obtained, alternative intermediate operations before the next operation, namely chromatography, are applied for example: decarboxylation with neutral cannabinoid as a targeted component, winterization in the presence of undesirable wages; liquid-liquid extraction in the presence of water-soluble undesirable substances in the extract; flushing through an adsorbent and/or molecular sieve, and/or glass material, and/or sand, in the presence of polar and/or non-polar undesirable, and/or targeted substances; filtration or combination thereof.
